(19) United States
(12) Patent Application Publication
(10) Pub. No.: US 2006/0235318 A1
(43) Pub. Date: Oct. 19, 2006

(54) Bipolar, Non-Vectorial Electrocardiography

(75) Inventor: Jorge Hernando Ordonez Smith, Columbia, MD (US)

Correspondence Address: JORGE HERNANDO ORDONEZ-SMITH, 5955 CAMELBACK LANE, COLUMBIA, MD, 21045, US

(73) Assignee: Jorge Hernando Ordonez Smith, Columbia, MD (US)

(21) Appl. No.: 11/163,140

(22) Filed: Oct. 6, 2005

Related U.S. Application Data
(63) Continuation-in-part of application No. 09/361,329, filed on Jul. 27, 1999, now abandoned.

(51) Int. Cl. A61B 5/04

(52) U.S. Cl. 600/509

(57) ABSTRACT

The present invention consists of analyzing the changes of electrical potential that occur in the body in synchronization with the contraction of the heart generated by the conduction of the monophasic electrical potentials occur during the contraction of the different structures that comprise the heart by the different muscular masses that are in close contact with each of them and a method to obtain EKGs by simultaneously recording from sixteen to over one hundred leads placing one “Common or Positive Electrode” on one of the extremities and from sixteen to a hundred “Exploring or Negative Electrodes” on the cephalic two thirds of the torso, close to the heart. In the “Bipolar” mode the “Common or Positive Electrode” is placed, preferably, on the distal third of the left leg. In the “Unipolar” mode, the “Common or Positive Electrode” is connected to an extra-corporeal “Constant Value Electrode”.

### CROSS-REFERENCE TO RELATED APPLICATIONS

This application is a continuation-in-part to the application Ser. No. 09/361,329, filed Jul. 27, 1999, now abandoned.

### OTHER REFERENCES

Einthoven, W.: Le Télécardiograme. Arch. Intern. Physiol. 1906; 4: 132-164
Einthoven, W.: The Different Forms of the Human Electrocardiogram and Their Signification, Lancet, 1912; 1: 853-861
Einthoven, W., Fahr, G., de Waart, A.: On the Direction and Manifest Size of the Variations of Potential in the Human Heart and on the Influence of the Position of the Heart on the Form of the Electrocardiogram, Pflüger's Arch. F Physiol., 1913; 150: 275-315
Goldberger, E.: A Simple, Indifferent, Electrocardiographic Electrode of Zero Potential and a Technique of Obtaining Augmented, Unipolar, Extremity Leads, Am. Heart J., 1942; 23:483-492
Katz, L. N., and Korey, H.: The Manner in Which the Electric Currents Generated by the Heart Are Conducted Away. Am. J. Physiol. 1935; 111: 83-90
Lewis, T.: Interpretations of the Initial Phases of the Electrocardiogram with Special Reference to the Theory of “Limited Potential Differences”, Arch. Int. Med., 1922; 30: 269-285
Ordóñez-Smith, J. H.: Study on the theories of: “Einthoven's Equilateral Triangle”, “Wilson's Central Terminal” and the “Unipolar Leads of Goldberg and Wilson”, Rev. Col. Cardiol., 2000; 8: 139-150
Supplementary Report by the Committee of the American Heart Association for the Standardization of Precordial Leads, Am. Heart J., 1938; 15: 235-239
Waller, A. D.: The Electromotive Properties of the Human Heart, Brit M. J., 1888; I: 751-754.
Waller, A. D.: On the Electromotive Changes Connected with the Beat of the Mammalian Heart and of the Human Heart in Particular, Phil. Trans. Roy. Soc. B., 1889; 180: 169-194
Wilson, F. N., Johnston, F. D., Macleod, A. G., Barker, P. S.: Electrocardiograms That Represent the Potential Variations of a Single Electrode, Am. Heart J., 1934; 9: 447-458
Wilson, F. N., Johnston, F. D., Rosenbaum, F. F., and Barker, P. S.: On Einthoven's Triangle, the Theory of Unipolar Electrocardiographic Leads, and the Interpretation of the Pericardial Electrocardiogram, A. Heart J., 1946; 32: 277-310

### REFERENCE TO A MICROFICHE APPENDIX

Not applicable

### STATEMENT REGARDING FEDERALLY SPONSORED RESEARCH

Not applicable

### BACKGROUND OF THE INVENTION

US patent Class: 600/509, 516, 517, 519, 523

IPC: A61 B/0402

1) Field of the Invention

The present invention relates to the acquisition of electrocardiographic recordings to facilitate the recognition of cardiac pathology and the genesis of such anomalies based in the discovery that today's accepted hypothesis of the genesis of such changes are erroneous.

2) Description of the Related Art

Augustus Dèsirè Waller (Waller, 1888 and 1889) did the first human electrocardiogram by immersing both hands of his assistant in containers of water and connecting them to a mercury electrometer. Initially, cardiograms were recorded using this technique. Only the hands, the feet, and the tongue were used to measure the differences in potential. As the science of electrocardiography improved, the containers were filled with saline and only the extremities were utilized using a labeling system still in use today; R for the right hand, L for the left hand, and F for the left foot.

Later Wilhelm Einthoven M. D. invented the string galvanometer, (Einthoven, 1906) and was able to obtain more accurate recordings. The terminology, established by A. D. Wailer M. D. for the different deflections produced by the heart, was changed by Einthoven in 1912 (Einthoven, 1912). Einthoven changed Waller's auricular deflection a to “P wave”, Waller's ventricular component V1 to “QRS complex”, and Waller's ventricular component V2 to “T wave”. Additionally he named a third ventricular component the “U wave”. This nomenclature is still in use today. Einthoven, Fahr, and de Waart (Einthoven et all, 1913) demonstrated the mathematical relationship, LIII=LII−LI, between the three standard leads and introduced the schema of the Equilateral Triangle to explain and calculate the changes that occur in the electrical axis of the heart. Sir Thomas Lewis's “Theory of Limited Potential Differences” (Lewis, 1922) strongly supported Einthoven's hypothesis by explaining how the different waves of the QRS Complex were generated.

F. N. Wilson, F. D. Johnston, F. D. Macleod, and P. S. Barker (Wilson et al, 1934) published the technique of obtaining unipolar leads based on Einthoven's hypothesis, controversy surrounding the genesis of the electrocardiogram was virtually non existent. E. Goldberger (Goldberg, 1942) discovered, while recording Unipolar V Leads of the extremities, that by disconnecting the extremity that was going to be recorded from “Wilson's Central Terminal” the shape of the lead did not change, but the amplitude was greater. He called this leads Augmented Unipolar Leads, or aV Leads. When F. N. Wilson, F. D. Johnston, F. F. Rosembaum and P. S. Barker (Wilson et al, 1946) published their Theory of Unipolar Leads, the hypothesis of Einthoven's “Equilateral Triangle” with its “Central Dipole” became the standard genesis of the electrocardiogram. Wilson et all. stated that by joining the electrodes of the three extremities, through high resistances to form a common electrode, the potential of this terminal was equal to or very near zero throughout the entire cardiac cycle. By coupling this Central Electrode to an exploring electrode placed in any area of the body the electrocardiographic trace would show only the changes in potential occurring in that area of the body.

During the early years of electrocardiography, there was a lack of consensus as to which leads to utilize and what should be considered a normal electrocardiogram. The disagreement was due to the plethora of theories pertaining to how the changes in potential were produced and to the near infinite number of different recordings that are labeled normal. Consequently, medical societies of different countries (AMA, 1938) created a Standard of Electrocardiographic Leads which remains unchanged today.

Today's EKG consists of obtaining the three standard leads used by Einthoven (Lead I, Lead II, and Lead III), the three Augmented Unipolar Leads of Goldberger (aVr, aVI and aVf), and the six Unipolar Precordial Leads of Wilson (V1, V2, V3, V4, V5, and V6). Of the twelve leads recorded, six use distant sites (the three extremities) and six use proximal and distal sites (the precordial area and “Wilson's Central Terminal”). The polarity of the different leads changes without any logic or explanation, the left leg (F) is connected to the positive terminal of the amplifier in Lead II, Lead III, and aVf, and is connected to the negative terminal in aVr, aVI, and in all the six Precordial Leads. The right arm (R) is always connected to the negative terminal, except in aVr when it is connected to the positive terminal. The left arm (L) is always connected to the negative terminal except in aVI and Lead I, when it is connected to the positive terminal. These twelve leads are interpreted mainly by patterns, and from comparison to previous electrocardiograms and/or changes in serial electrocardiograms. In standard medical practice a definitive diagnosis can be reached only by taking in consideration, serial recordings, the clinical picture, and other auxiliary tests (radiological, chemical, endoscopical, etc). Abnormal pattern changes alone are not diagnostic and can, in fact, be present in perfectly normal individuals. These changes are empirically related to cardiac pathology, as have been established by more than a hundred years of experimentation and practice.

A typical electrocardiographic trace is comprised of the following:
The P wave that represents the contraction of the auricle.
The QRS complex that represents the depolarization of the ventricles and consists of the waves Q, R, and S. In a normal complex, one, two or all, waves can be present.
The T wave that represents the repolarization of the myocardium.
The U wave seen is seen occasionally and has no clear genesis.
   Personal Research

In personal research, I have found that:
(1) The addition of the values of the three standard leads is equal to zero due to the mathematical axiom,
   a) If a−b=x, b−c=y, and c−a=z, then a+b+c=0
and not due to the validity of Einthoven's “Equilateral Triangle”, (Ordóñez-Smith, 2000).
(2) The human body is not a “flat, homogeneous plate in the form of an equilateral triangle” (Einthoven, 1913). Rather, the monophasic changes of electrical potential that occur in the surface of the body in synchronicity with the contraction of the heart are conducted throughout the body to the body surface in the manner proposed by Katz, L. N. and Korey, H., (Katz & Korey, 1935). That is, by the close contact of the different structures of the heart with the spinal musculature, the musculature of the anterior and lateral walls of the chest and abdomen, and the diaphragm.
(3) The heart does not generate the changes of electrical potentials in or on the body as a dipole localized in “A small spot, H, in the middle of the triangle” (Einthoven, 1913). In fact the auricles and the P wave are most prominent in:
   The right and left supra-clavicular areas.
   The supra-sternal notch.
   The right anterior chest wall area.
   The QRS complex is most prominent in:
   The caudal two thirds of the anterior and lateral surfaces of the left hemi-thorax.
   The cephalic two thirds of the anterior surface of the right hemi-thorax.
   The T wave is most prominent in:
   The anterior surface of the right chest wall.
   The anterior surface of the left chest wall.
   The changes of potential that are present in the right ventricle are most prominent in:
   The upper anterior chest wall pathway.
   The changes in the antero-lateral surface of the ventricles are most prevalent in:
   The anterior and left lateral chest walls and abdominal wall pathway.
   The changes in the posterior surface of the left ventricle are most prevalent along
   The spinal musculature.
(4) The potential of “Wilson's Central Terminal” is:
   b) P_{(RLF)}=F+(LI+LII/3),
the potentials of Goldberger's “Augmented Terminals” are: for aVf,
   c) P_{(RL)}=F+(LII+LIII/2),
for aVr,
   d) P_{(LF)}=F+(LIII/2), and
for aVI,
   e) P_{(FR)}=F−(LII+LIII/2).
In these equations, P_{(RLF)t} is the value of the electrical potential of “Wilson's Central Terminal”, and P_{(RL)}, P_{(LF)}, and P_{(FR)}, are the values of the electrical potentials of the three “Central Terminals” of Goldberger. From equations b, c, d, and e it is clear that the potentials of these terminals are not equal to zero or near zero as Wilson, F. N. et all and Goldberger concluded.
(5) The so-called “Unipolar” leads are not “Unipolar”, but complex “Bipolar” leads. They do not represent the true changes in potential that are occurring at the sites where the electrodes are placed. In reality, as long as all the electrodes are placed on or in the body, no true “Unipolar” leads can be recorded.
(6) From a mathematical point of view the three standard leads of the electrocardiogram are the first derivatives of three variable functions,
   f) fR, fL, and fF
(7) The variable functions represent the changes of electrical potential generated by the monophasic potentials of the myocardium in each one of the different areas on the surface of the leg and both arms. In these variable functions the X-axis represents time in milli-seconds, and the Y-axis represents electrical potential in milli-volts.
(8) The difference in amplitude, morphology and timing between the monophasic electrical potentials generated by the contraction of the different components of the myocardium and their conduction throughout the body by the muscular masses that are in close contact with them is the genesis of the different waves of an electrocardiographic trace.
(9) The Unipolar Leads of Wilson and the Augmented Unipolar Leads of Goldberger even accepting their value of zero are recording negative potentials at the apex of the myocardium and at the left leg, and not the supposedly positive potentials that exist in those areas.

### SUMMARY OF THE INVENTION

The invention is based on the realization that “Einthoven's Law” is valid because it fulfills the mathematical axiom,
a) If a−b=x, b−c=y, and c−a=z, then a+b+c=0
   and not due to the validity of Einthoven's “Equilateral Triangle”. Placing three electrodes in any area of the body and recording an electrocardiogram will fulfill “Einthoven's Law”.

Today's accepted hypothesis about the genesis of electrocardiography, the hypothesis of Lewis's Limited Potential Differences that explains the genesis of the QRS complex, Wilson's Central Terminal to obtain Unipolar Leads, and Goldberger's three Central Terminals to obtain Augmented Unipolar Leads, are totally depended in the absolute validity of the hypothesis of Einthoven's Equilateral Triangle with the Central Dipole. Since Einthoven's hypothesis is unsustainable in view of the new finding, every hypothesis that depends on its absolute validity to be true becomes unsustainable too.

The new bipolar, non-vectorial electrocardiography recognizes three facts:
(1) The different waves of an electrocardiographic trace, the P wave, the QRS complex, the T wave, and the U wave, are generated by the differences in amplitude, morphology and timing of the monophasic electrical potentials generated by the contraction of the different structures of the myocardium and their conduction throughout the body by the muscular masses that are in close contact with them.
(2) The monophasic potentials generated by the contraction of the heart do not propagate to the surface and throughout the body as if generated by a Central Dipole but by the close contact of the different structures that comprise the heart with the musculature of the chest and abdominal walls, the diaphragm and the spine.
(3) Any electrocardiographic lead obtained by placing the electrodes in or on the body is a bipolar lead.

The new method consists of obtaining from twelve to a hundred electrocardiographic leads by using as many identical individual amplifiers and pairing the negative terminal of each amplifier to an “Exploring or Negative Electrode” and the positive terminal to a “Common or Positive Electrode”. The “Common or Positive Electrode” is placed on one leg or the right arm to obtain “Bipolar” electrocardiograms or on an extracorporeal “Constant Value Electrode” to obtain “Unipolar” electrocardiograms. The number of leads selected will depend on whether the recorder is to be used as a small unit, (doctor's office, portable unit, etc.) or as a large unit for body mapping (cardiology center, hospital). For small units the “Exploring or Negative Electrodes” are placed on: the anterior and left lateral surfaces of the chest wall, on the posterior and superior surfaces of both shoulders, and on the inter-scapular area. The placement of the electrodes for the body mapping units are done from the base of the neck down to the waist on the anterior and lateral surfaces of the chest and upper abdomen, and on the back from T-1 level on down to L-2 level.

The use of the same variable function (The changes in electrical potential in either leg, or the right arm, or the extracorporeal Constant Value Electrode) as the positive terminal on all the amplifiers allows for the information obtained being more evident and easier to analyze than the information generated from today's traditional standard electrocardiograms. In the “Bipolar” mode, the values generated by either leg or the right arm, are calculated and subtracted from all the leads. The final tracing will report of the values generated by each of the “Exploring or Negative Electrodes”. The digital data sets obtained by the recorder are to be saved on a “Digital Disk” that will be part of the permanent report. The final tracing in the “Unipolar” mode will be the real changes of electrical potential of each Exploring or Negative Electrode. When subsequent EKGs are recorded, the stored identified digital data sets from previous EKGs are to be retrieved and compared by the recorder with the newly obtained identified digital data sets. The recorder will report any changes obtained and report them together with the new electrocardiogram.

### OBJECTIVES AND FEATURES OF THE INVENTION

It is an objective of the present invention to obtain data that are more reliable and characteristic of the electrical potential differences generated by the contraction of the myocardium in its normal and abnormal states.

It is a further objective of the present invention to provide a method of enhancing and facilitating the recognition of the changes of electrical potential differences on the body surface that are pathognomonic in the presence of myocardial pathology.

It is a further objective of the present invention to provide a method of analysis of the different changes of electrical potential on the surface of the body to facilitate the recognition of normal and abnormal electrocardiogram patterns.

It is a feature of the present invention to acquire the changes of electrical potential on the surface of the body that occur in synchronization with the contraction of the heart at sites that are closer to the heart and in the areas where each wave of the electrocardiographic trace is more prominent.

It is a further feature of the present invention to analyze the changes of electrical potential on the surface of the body that occur in synchronization with the contraction of the heart as a result of characteristic conduction patterns of the monophasic electrical potentials generated by the different structures of the myocardium toward the body surface.

It is a further feature of the present invention to preserve, on a Digital Disk, the electrocardiographic digital data sets, including the subject's identification and the exact anatomical placement of the electrodes used for the electrocardiographic recording.

It is a further feature of the present invention to compare the stored identified electrocardiographic digital data sets with the newly obtained identified electrocardiographic digital data sets.

It is a further feature of the present invention to report any differences between the two sets of identified electrocardiographic digital data sets and to save both sets of data for further evaluations.

### DESCRIPTION OF THE DRAWINGS

This invention and its advances over the prior art can best be understood by reading the specification which follows in conjunction with the drawings herein, in which; According to one embodiment of the present invention:

FIG. 1 is a block diagram of an electrocardiographic method in which the “Common or Positive Terminals” of amplifiers 1 to n are connected to an electrode placed on one of the legs of the subject. The “Exploring or Negative Terminals” are connected to electrodes placed on the cephalic two thirds of the subject's torso.

FIG. 2 is a master flow chart for the Microprocessor's different stages.

FIG. 3 is a block diagram of an Electrocardiographic method in which the “Common or Positive Terminals” of the amplifiers 1 to n are connected to a “Constant Value Electrode” and the “Exploring or Negative Terminals” of the amplifiers 1 to (n−1) are connected to electrodes placed on the cephalic two thirds of the subjects torso and the “Exploring or Negative Electrode” of amplifier n is connected to an electrode placed on one of the subject's leg.

To emphasize the difference between the present invention and the standard leads of today's electrocardiographic art, said leads are schematized in the figure of the subject.

### DETAILED DESCRIPTION OF THE INVENTION

The purpose of the present invention is to record the changes of electrical potential that occur on the body in synchronization with the contraction of the myocardium where they are more obvious. It differs from today's art by recognizing four findings:
(1) “Einthoven's Law” is valid because it fulfills the premises of the mathematical axiom, “If a−b=x, b−c=y and c−a=z, then x+y+z=0”, (Ordóñez-Smith, 2000). Placing three electrodes anywhere in the body and recording an electrocardiogram will fulfill “Einthoven's Law”.
(2) The heart does not behave like a dipole at any instant during the cardiac cycle. The monophasic changes of electrical potentials that occur in or on the body in synchronization with the heart beat are conducted from the heart to the surface through three distinct muscular pathways: the para-espinal musculature, the diaphragm and the musculature of the anterior and lateral surfaces of the chest and abdomen.
(3) The different waves of an electrocardiographic trace are the result of the differences between the amplitude, morphology, and timing of the monophasic electrical potentials generated by the contraction of the different structures of the myocardium and their conduction throughout the body by the different muscular masses that are in close contact with them.
(4) Connecting the positive and negative terminals of the amplifiers to two, (Lead I, Lead II and Lead III), three, (aVr, aVI and aVf), or four electrodes, (V1, V2, . . . V6), that are placed on or in the body will always give a bipolar lead (Ordóñez-Smith, 2000).

The placement of the electrodes differs from the traditional electrocardiograms. They are to be placed on the subject's cephalic two thirds of the torso and are identified by their anatomical placement. Placement is identified by the use of easily recognizable anatomical reference points on the anterior and posterior surfaces of the body and by the distance from those points, whether it is medial, transversal, on the right side or on the left side. On the anterior surface of the body, the points of reference are: a) the supra-sternal notch, b) the inter-costal spaces and c) the xiphoid. On the posterior surface they are: a) the spinal process of the sixth cervical spine and b) the inter-vertebral spaces of T1-T2 to T12-L1. The measurements are done from the medial line along lines that intersect the reference points perpendicularly. The medial axilar line is the limit between anterior and posterior surfaces of the body. On the anterior surface two measurements should be included for electrodes placed above the sternal notch or below the xiphoid. They are the distance from the reference point to the point where the medial line is transected by the perpendicular line that passes through the electrode, and the distance from said point in the mid-line to the electrode site.

The embodiments according to the present invention will now be described in detail with reference to the drawings. The different electronic components described in the embodiments, preamplifiers, amplifiers, A/D multiplexers, digital filters, calculators, analyzers, digital disks, modems, keyboards and printers are commercially available components.

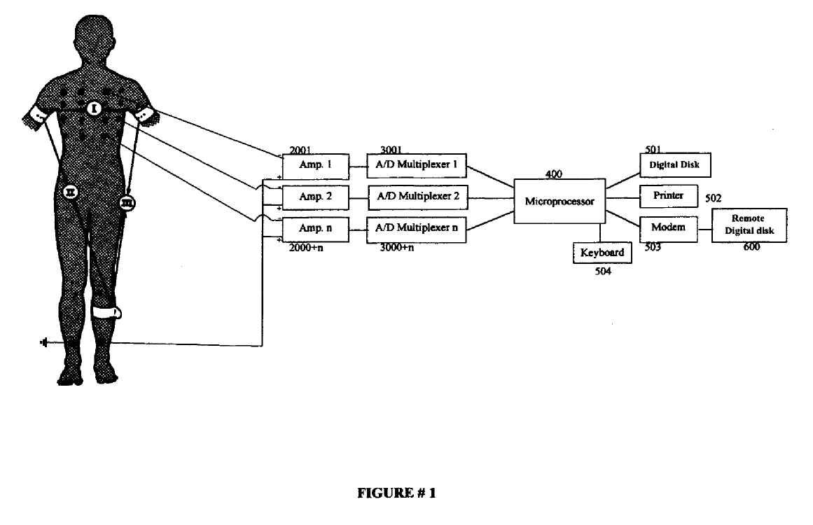

FIG. 1 shows a first embodiment of the present invention. As shown, the subject is positioned so that the cephalic two thirds of his torso is connected, through the desired number of electrodes n, to the “Exploring or negative Terminal” of amplifiers 1 to n and the left leg is connected to the “Common or positive Terminal” of amplifiers 1 to n to generate a “bipolar” electrocardiogram or first derivative. The figure, for simplicity, shows only three electrodes placed on the subject's chest and one electrode placed on the distal third of his left leg. A “Ground Electrode” is placed on the right leg to reduce the noise. Each high-gain, low-noise amplifier (2001−2000+n) has an input isolation switch to prevent current leakage to the subject.

Each amplifier is connected to its own individual Analog-to-Digital Multiplexer (3001−3000+n). The multiplexer will sample the n amplified analog bipolar electrocardiograms or first derivatives at a rate of around 100,000 samples per second with 12-bit resolution to generate n digital data sets. The digital data sets are fed to the Microprocessor (400).

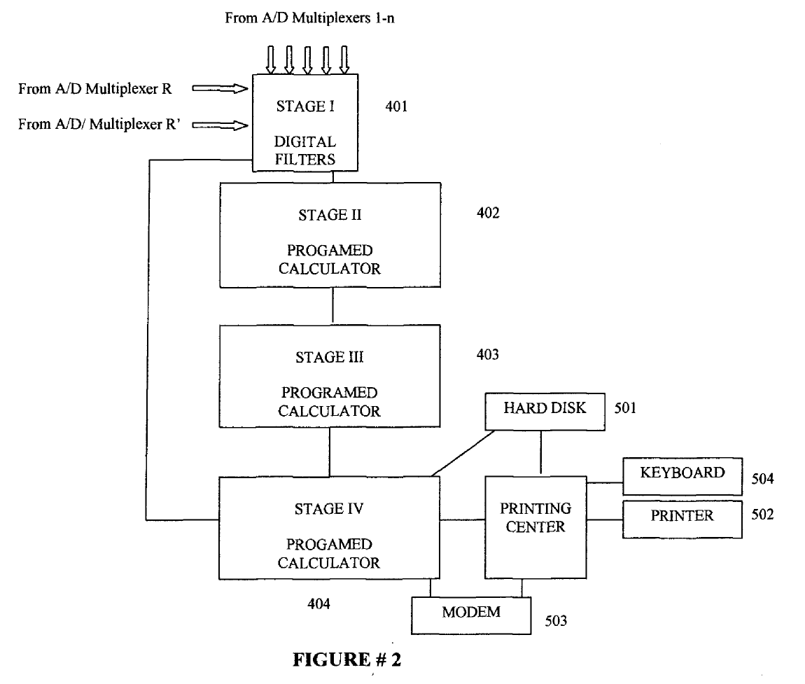

FIG. 2 shows the flow through the Microprocessor's different stages.
(1) The first stage is a Digital Filter (401) with two-band pass filters between 0.5-55 Hz and 65-1000 Hz and band stop filters between 55-65 Hz and all frequencies below 0.5 Hz and above 1000 Hz. The n filtered digital data sets are forwarded to the second, third and fourth stages of the microprocessor.
(2) The second stage, comprised of a programmed calculator (402), randomly pairs the filtered digital data sets and subtracts said sets from each other to obtain n/2 digital data sets of the second derivatives.
(3) The second derivative digital data sets are fed to the third stage, comprised of an analyzer (403). In this stage the n filtered digital data sets of the first derivative are compared with the n/2 digital data sets of the second derivative to obtain the approximate values generated by the electrode placed on the leg of the subject. The digital data sets of the values generated by the leg electrode are fed to the fourth stage.
(4) The fourth stage, comprised of a programmed calculator and a data analyzer (404), subtracts the digital data set of the values generated by the leg from the n digital data sets of the first derivative, the difference giving a good approximation of the values generated by each individual electrode. All the n digital data sets of the values generated by each electrode and the digital data set of the values of the electrode on the leg are fed to the data processor.
(5) In the fifth stage, comprised of a data processor (405), the operator identifies the n digital data sets of the first derivatives and the n+L digital data sets of the values generated by each individual electrode by the anatomical placement of each Exploring or Negative Electrode and the placement of the Common or Positive Electrode.
(6) If there are no previous electrocardiograms, the digital data sets are fed to: the printer to print the electrocardiogram, the disk drive and/or the modem to save the identified electrocardiographic digital data sets of the subject on a digital disk.
(7) If there are previous electrocardiograms, the stored identified electrocardiographic digital data sets of the previous electrocardiograms are retrieved from the digital disk and fed to the microprocessor's fourth stage analyzer to find if there are differences between the present and prior electrocardiograms.
(8) If no changes are found no new digital data sets are generated.
(9) If there are changes, the changes will be reported in new digital data sets are fed to the Data Processor to be printed and stored in a digital disk.

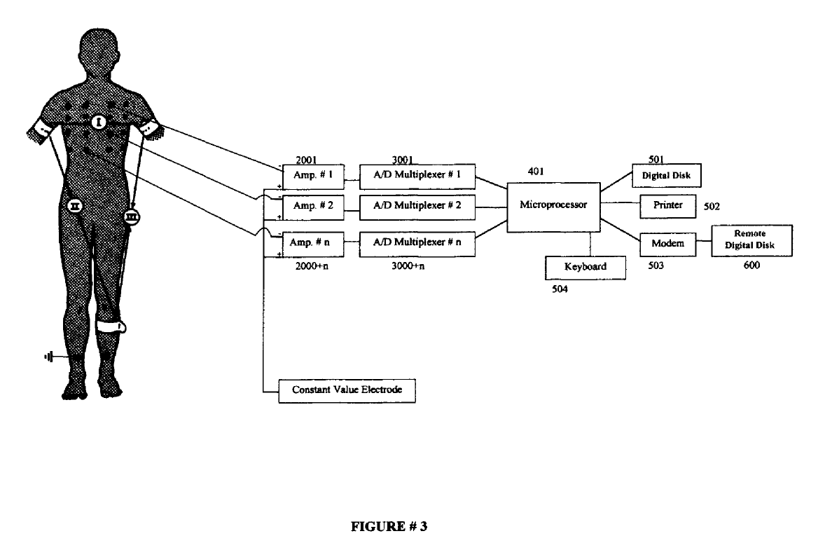

FIG. 3 shows a second embodiment of the present invention. To generate “unipolar” electrocardiograms the subject is positioned so that the cephalic two thirds of the torso and the leg are connected through electrodes to the desired number of “Exploring or Negative Terminals” and the “Common or Positive Terminal” of amplifiers 1 to n and F are connected to a “Constant Value Electrode”. The figure is simplified to show only three electrodes: 1, 2 and n.
(1) The electrodes are connected to the “Exploratory or negative Terminal” of each individual high-gain, low-noise, input-switch-insulated amplifiers (20001 to 2000+n). The positive terminals of the amplifiers are connected to the “Constant Value Electrode”.
(2) The amplified analog electrocardiographic traces are fed to individual analog/digital multiplexers (3001 to 3000+n) that will sample them at a rate of 100,000 per second with 12-bit resolution.
(3) The first stage is a Digital Filter (401) with two-band pass filters between 0.5-55 Hz and 65-1000 Hz and band stop filters between 55-65 Hz and all frequencies below 0.5 Hz and above 1000 Hz.
(4) The n filtered digital data sets are forwarded to the fifth stage of the microprocessor comprised of a data processor (405). The operator identifies the filtered digital data sets by the anatomical localization of the Exploring or Negative Electrodes, the placement of the Common or Positive Electrode, and the subject's identification data. These identified filtered digital data sets are processed according to different commands from the operator.
(5) If there are no previous electrocardiograms, the digital data sets are fed to: the printer to print the electrocardiogram, the disk drive and/or the modem to save the identified electrocardiographic digital data sets of the subject on a digital disk.
(6) If the subject has a previous “bipolar” electrocardiogram, the filtered digital data sets 1 to n and F, are fed to the microprocessor's second stage programmed calculator (402) to individually subtract from them the filtered digital data set of the amplifier F to generate ‘bipolar’ electrocardiograms.
(7) These ‘bipolar’ filtered electrocardiograph digital data sets are feed into the next stages of the microprocessor to follow the process described in the previous embodiment.
(8) If the previous electrocardiogram was “unipolar”, the identified electrocardiograph digital data sets retrieved from the digital disk are fed to the microprocessor's fourth stage. Said stage's analyzer compares the previous sets of unipolar identified electrocardiograph digital data with the new sets of unipolar identified electrocardiograph digital data.
(9) The subsequent stages follow the steps 7, 8, and 9 described in the previous embodiment.

Since certain changes may be made in the above constructions without departing from the scope of the invention, it is intended that all matter contained in the above description be interpreted as illustrative and not limiting in any way. It is also to be understood that the following claims are to cover all generic and specific features of the invention described herein, and all statements of the scope of the invention which, as a matter of language, might be said to fall there between.

Definition List 1

| Term | Definition |
|---|---|
| “Unipolar” | Measurements between terminal pairs |
| | when one terminal is connected to a |
| | “Constant Value Electrode” an the other |
| | is connected to an electrode placed on |
| | the subject. |
| “Bipolar” | Measurements between terminal pairs |
| | when both terminals are connected to |
| | electrodes placed on the subject. |
| “Ground Electrode” | Electrical connection to the ground. |
| “Constant Value | Electrode connected to an element of |
| Electrode” | known electrical potential that is |
| | constant and free of interference from |
| | the electrical fields of the subject and |
| | the environment. |
| “Value” | Electrical potential difference between |
| | amplifier terminal pairs. |
| “Exploring or Negative | Negative terminal of the individual |
| Terminals” | amplifiers. |
| “Common or Positive | Positive terminal of the individual |
| Terminals” | amplifiers. |
| “Exploring or Negative | Electrodes connected to the negative |
| Electrodes | terminal of the amplifiers and placed on |
| | the subject's torso. |
| “Common or Positive | Electrode connected to the positive |
| Electrode” | terminal of the amplifiers and placed on |
| | the distal third of either leg or right arm. |
| “Electrocardiographic | Difference between the electrical pairs of |
| Lead” | each individual amplifier and identified |
| | by the anatomical site of the “Exploring |
| | or Negative Electrode” in the subject's |
| | torso. |
| “Digital disk” | Systems used to store digital data. |
| | Floppy disk, CD, Hard disk, DVD, etc. |

What is claimed is:

1. A method of analyzing the changes of electrical potential that occurs in or on the body in synchronization with the heart beat as bipolar leads generated by the conduction of the monophasic electrical potentials of the different structures that form the myocardium by the direct contact of these structures with the muscular masses that are in close contact with them.

2. A method of analyzing the P wave, the QRS complex, the ST segment, the T wave, and the U wave of the electrocardiogram as generated by the differences in amplitude, morphology, and timing of the monophasic potentials generated by the different structures of the myocardium and conducted to the different areas of the body by the muscular masses that are in close contact with them.

3. A method of claim 1 and claim 2 wherein the changes of potential on or in the body that occur simultaneously with the contraction of the heart are registered to obtain: “Bipolar” electrocardiograms by connecting the “Exploring or negative terminal” and the “Common or positive terminal” of the amplifier to electrodes placed in the surface of the body to detect the changes of potential on the surface of the body that occur in synchronicity with the contraction of the heart, based on the fact that said changes are conducted from the myocardium to the surface through three mayor muscular masses; the spinal musculature, the diaphragm and the musculature of the chest and abdominal walls. Said method comprising of:
1) Placing, from 16 to 100 electrodes on the cephalic two thirds of the subject's torso, one on the distal third of the either of the legs or right arm, and one ground electrode on any area of the body.
2) Amplifier means connected to the electrodes to obtain analog electrocardiographic amplified signals pairing the negative terminal of each individual low-noise, high-gain, input switch isolated amplifier to its own Exploring or Negative Electrode placed on the subject's torso, and the positive terminal of all the amplifiers to the Common or Positive Electrode placed in the subject's legs or right arm.
3) Analog/digital conversion means connected to the amplifier means to transform each amplified analog electrocardiographic tracing to sets of digital data.
4) A band-pass band-stop digital filter means connected to the analog/digital means to improve the signal-to-noise ratio in the electrocardiographic digital data sets.
5) A calculator means connected to the digital filter means to obtain the difference between the filtered electrocardiographic digital data sets by pairing them, at random, to obtain the second derivative digital data sets,
g) f″(1-6), f″(2-12), . . . f″(14-n).
6) A data analyzer means connected to the calculator means and the digital filter means to analyze the differences between the values of the second derivative digital data sets and the filtered electrocardiographic digital data sets of the first derivative. The values not found to be common between the two sets of derivatives are reported as values originated from the Common or Positive Electrode.
7) A calculator means connected to the data analyzer means to subtract from the filtered electrocardiographic digital data sets the digital data sets of the values originating from the Common or Positive Electrode.
8) Calculator means connected to the digital filter means, the digital disk means and the modem means to obtain “bipolar” filtered electrocardiographic digital data sets.
9) Data analyzer means connected to the digital filter means, the calculator means and the digital disk means to obtain the differences between the current filtered electrocardiographic digital data sets and the previously saved identified electrocardiographic digital data sets.
10) A data processor means connected to the data analyzer means and calculator means to identify each set of electrocardiographic digital data by the anatomical placement of its Exploring or Negative Electrode and the identification data of the subject.
11) A digital disk drive means connected to the data processor means and the data analyzer means to save or retrieve identified electrocardiographic digital data sets.
12) A printer means connected to the processor means to print the electrocardiogram.
13) A modem means connected to the data processor means and the data analyzer means to retrieve from or save on a remote digital disk the identified electrocardiographic digital data sets.
14) A keyboard means connected to the data processor means for the operator to submit the information to identify the electrocardiographic digital data sets and give the commands to store or retrieve the identified electrocardiographic digital data sets.

4. A method of claim 3 wherein “Unipolar” electrocardiograms are obtained by the detection of the changes of potential in the body surface that occur in synchronicity with the contraction of the heart. Said method comprising of the same elements 1) to 14) as the previous embodiment and of:
15) A Constant Value Electrode means connected to amplifier means to obtain “unipolar” electrocardiographic leads.

5. A method of claim 3 and claim 4 wherein the areas of placement of the “Exploring or Positive Electrodes” are selected to gather information, on each of the different waves that comprises the electrocardiogram, according to the areas where they are more prominent and obvious.

6. A method of claim 3 wherein the values generated by the electrode placed in the subject's leg or right arm are calculated by:
1) Randomly pairing the values of the first derivatives,
h) f′(1-F), f′(2-F), . . . f′(n−F).
2) Subtracting the paired first derivatives to obtain the second derivatives,
i) f″(1-F)-(12-F), f″(6-F)-(20-F), . . . f″(n-F)-(14-F).
3) Analyzing the values of the second derivatives against the values of the first derivatives to obtain the values that are present in the first derivatives but are absent in the second derivatives to obtain the values generated by the leg or arm,
j) fF_{g} or fR_{g}.

7. A method of claim 3 and claim 6 wherein the values generated by the Common or positive Terminal” connected to the electrode placed in the subject's leg or right arm are subtracted from the values of the first derivatives 1 to n,
k) f(1-F_{g}), f (2-F_{g}), . . . f(n-F_{g}).
The results of the subtractions are to be reported in the final graphic as the values generated from electrodes 1 to n.

8. A method of claim 3, claim 4, claim 6, and claim 7 wherein the final report of the electrocardiogram, beside the standard graphic, will include all the identified electrocardiographic digital data sets, obtained by the recorder, saved in a digital disk. This will allow the operator, in the future, to obtain new electrocardiograms placing the electrodes in the same anatomical sites as the original electrocardiogram and obtain meaningful comparisons, by the recording equipment, between the newly obtained electrocardiographic digital data sets and the previously saved identified electrocardiographic digital data sets.

9. A method of claim 3 and claim 8 wherein changes found between the previously saved identified electrocardiographic digital data sets and the new electrocardiographic digital data sets are shown in the final graphic report.
